(19) United States
(12) Patent Application Publication
(10) Pub. No.: US 2025/0290909 A1
(43) Pub. Date: Sep. 18, 2025

(54) ARRANGEMENT COMPRISING A PORTABLE GAS DETECTION DEVICE AND A STATIONARY GAS DETECTION DEVICE

(71) Applicant: Dräger Safety AG & Co. KGaA, Lübeck (DE)

(72) Inventors: Günter Wahlbrink, Lübeck (DE); Timo Schuster, Lübeck (DE)

(21) Appl. No.: 19/081,786

(22) Filed: Mar. 17, 2025

(30) Foreign Application Priority Data
Mar. 18, 2024 (DE) 102024107646.1

(51) Int. Cl. G01N 33/00

(52) U.S. Cl. CPC G01N 33/0075; G01N 33/0063

(57) ABSTRACT

An arrangement and method for monitoring a spatial area for the presence of a target gas are described. A user may wear a portable gas detection device on his/her protective equipment. A stationary device may be positioned at an installation location. Both devices may comprise a respective sensor unit and have a respective detection area. The sensor unit may measure the target gas concentration in the detection area. The target gas concentration measured by the stationary device may be transmitted to the portable device. An evaluation unit of the portable device may automatically determine whether the target gas concentration in one of the two detection areas is outside a specified concentration range. If this is the case, an output unit of the portable device may output an alarm in a form perceptible to a human.

### CROSS-REFERENCE TO RELATED APPLICATIONS

This application claims the priority of German Patent Application No. 102024107646.1, filed on Mar. 18, 2024, and titled “ARRANGEMENT COMPRISING A PORTABLE GAS DETECTION DEVICE AND A STATIONARY GAS DETECTION DEVICE,” which is hereby incorporated by reference in its entirety for all nonlimiting purposes.

### SUMMARY

The present disclosure relates to an arrangement having at least one portable gas detection device and at least one stationary gas detection device. The arrangement may monitor a spatial area for the presence of a gas and warn a user of the portable gas detection device if the concentration of this gas is outside a specified range, in particular if it is too high or too low. This gas will hereinafter be referred to as the “target gas.” Furthermore, the disclosure relates to a method for monitoring the spatial area using such an arrangement.

A portable gas detection device which a user can attach to his/her personal protective equipment has become known. The portable gas detection device measures the concentration of the target gas and warns the user if the area around him/her contains the target gas at a concentration which is harmful to humans.

The object addressed by the disclosure is that of providing an arrangement comprising a portable gas detection device, wherein the arrangement can more reliably warn a user of the portable gas detection device of an impermissible concentration of a target gas in a spatial area to be monitored than known arrangements can do. Furthermore, the object addressed by the disclosure is that of providing a method for monitoring a spatial area for the presence of a target gas, wherein the method is intended to be more reliable than known methods of this type.

The object is achieved by an arrangement having the features described herein. Advantageous embodiments are specified in the claims. Wherever it makes sense, advantageous embodiments of the arrangement according to the disclosure are also advantageous embodiments of the method according to the disclosure, and vice versa.

The arrangement according to the disclosure comprises a first portable gas detection device and a first stationary gas detection device. The method according to the disclosure is carried out using such an arrangement.

In the following, the term “device” will be used for short. Unless otherwise specified, this refers to a gas detection device.

A user can carry the first portable device with him/her when staying in a spatial area. Preferably, the first portable device can be attached to the user's personal protective equipment. The first stationary device can be positioned at or in an installation location.

In addition, the term “target gas” will be used below. The target gas can be a gas which is harmful to humans or a gas mixture containing at least one such harmful gas, in particular a flammable and/or toxic gas, for example methane or carbon monoxide. The target gas can also be oxygen or another gas which humans require or also an anesthetic.

A first concentration range is specified. If the concentration of the target gas surrounding a person is outside the first concentration range, this target gas concentration may put the person at risk. The first concentration range therefore defines the permissible target gas concentration. A concentration outside the first concentration range is also referred to as an impermissible concentration. Preferably, the first concentration range defines an upper concentration limit (in particular for a harmful gas or an anesthetic) or a lower concentration limit (in particular for oxygen).

The first portable device comprises a sensor unit and has a detection area. The sensor unit is configured to measure the concentration of a target gas in the detection area of the first portable device. The detection area is an area (region, space) around the device, and the sensor unit provides a result for the target gas concentration in the detection area within a given time period. As a rule, the detection area moves with the user as the user with the first portable device moves through the spatial area. The detection area of the first portable device is preferably configured as follows: As long as the target gas concentration in the detection area of the first portable device is within the first concentration range, the user of the first portable device is currently not at risk from the concentration of the target gas.

Note: The following specifies that a sensor unit can measure the concentration of a target gas. This means the following: The sensor unit can measure at least one detection variable, wherein the measured detection variable or the combination of measured detection variables correlates with the desired target gas concentration. The detection variable is, for example, an electrical voltage or current or charge. The device to which the sensor unit belongs automatically derives the desired target gas concentration from the measured detection variable value or the combination of the measured detection quantity values.

It is possible that the sensor unit is able to measure the individual concentrations of a plurality of target gases which are simultaneously present or the sum of the target gas concentrations. For short, the term “target gas concentration” will be used below, and this also refers to the individual concentration of a plurality of target gases as well as the sum of the target gas concentrations.

The first stationary device also comprises a sensor unit and also has a detection area. This sensor unit can measure the concentration of a target gas in the detection area of the first stationary device. The detection area of the first stationary device can be located around the first stationary device or at a free end of a fluid-guiding unit through which a gas sample is drawn, e.g. sucked, into the interior of the first stationary device. For example, this free end is located in a container, while the first stationary device is located outside the container.

The detection areas of the two devices can have the same dimensions or differ from one another in at least one dimension. The detection area can be outdoors or in a container, depending on where the user of the first portable device is located or where the first stationary device is installed.

Both devices comprise a respective communication unit. A data connection is or can be at least temporarily established between the first portable device and the first stationary device, specifically by means of the two communication units. This data connection can be used to transmit at least one message from the first stationary device to the first portable device, and optionally also to transmit a message in the opposite direction. Preferably, the message is transmitted via radio waves, i.e. wirelessly, from one communication unit to the other communication unit. The method according to the disclosure comprises the steps of at least temporarily establishing this data connection and transmitting, via the data connection, the message from the first stationary device to the first portable device. Via this data connection, a message can be transmitted from the first stationary device to the first portable device, wherein this message comprises information about a target gas concentration measured by the first stationary device in its detection area. Optionally, the message also comprises information about the geoposition of the first stationary device.

The first portable device further comprises a signal-processing evaluation unit. The evaluation unit of the first portable device can automatically make the following two decisions:
Is the target gas concentration, which the first portable device has measured in its detection area, outside a specified first concentration range?
Is the target gas concentration, which the first stationary device has measured in its detection area, and which was transmitted to the first portable device, outside the first concentration range?

The evaluation unit of the first portable device is therefore able to compare the target gas concentration measured by the first portable device and the target gas concentration measured by the first stationary device with the same specified first concentration range.

The first portable device can generate an alarm in response to at least one of the following two events having occurred:
The target gas concentration measured by the first portable device in its detection area is outside the first concentration range.
The target gas concentration in the detection area of the first stationary device is outside the first concentration range.

The first portable device comprises an output unit. By means of this output unit, the first portable device can output the alarm, specifically in at least one form perceptible by a human, in particular visually, acoustically, and/or haptically (through vibrations). Outputting an alarm is understood as a message indicating that the target gas is present at an impermissible concentration, i.e. at a concentration outside the first concentration range. The first portable device is configured to cause the output unit to output the alarm if the alarm has been generated in response to the events just described. A user of the first portable device is able to perceive this alarm.

The first portable device can alert the user thereof if the target gas is present in the detection area of the first portable device at a concentration outside the first concentration range. Since the first portable device has its own output unit, it is possible, but thanks to the disclosure not necessary, for an alarm to be transmitted to an external receiver and for the external receiver to prompt the alarm to be output. The first concentration range can be designed in such a way that a user is currently not at risk if the target gas concentration in the entire detection area of the first device is in the first concentration range. Owing to the first portable device, the user is therefore warned of an impermissible target gas concentration in the detection area of the first portable device. Preferably, the response time of the first portable device is sufficiently short so that, if the target gas concentration increases, a user is warned of an impermissible target gas concentration quickly enough.

However, it is possible that an impermissible concentration of the target gas occurs, but this impermissible target gas concentration is at least temporarily outside the detection area of the first portable device and therefore cannot be detected by the first portable device. This can also put the user at risk. To be specific, it is possible that a harmful target gas may escape and that some of the escaped target gas may reach the user due to a natural or artificially generated air flow and/or due to overpressure. In addition, it is possible that the user moves together with the first portable device and then reaches the harmful target gas which has escaped, for example in an enclosed space. The same applies to an oxygen concentration which is too low. Although the first portable device will detect the impermissible target gas concentration, it should be borne in mine that the first portable device inevitably has a certain response time. The response time is the time period which elapses between the following two events:
The target gas reaches the detection area of the first portable device.
The first portable device outputs an alarm.

The response time may depend on the measuring principle used. In addition, it is often desirable to warn the user about the target gas as early as possible, for example with the aim that the user can complete a job he has started and does not have to interrupt it suddenly. The same applies if the target gas is important for a person, for example is oxygen, and there is a risk of the target gas concentration in the user's environment being too low.

According to the disclosure, the arrangement comprises, in addition to the first portable device, a first stationary device, optionally a plurality of stationary devices. The user or another person can position the or each stationary device in one respective installation location. Preferably, the installation locations are defined and used as follows: If a harmful target gas can escape from a location in the spatial area to be monitored or if a target gas necessary for humans must be present, this location lies in the detection area of the or at least one stationary device of the arrangement. Conversely, each detection area of a stationary device preferably comprises a location at which a target gas can escape. A location where a harmful target gas can escape is, for example, a valve or a junction or branching point of pipelines or a tank or other container containing a fluid which is harmful to humans or even a medical device from which an anesthetic can escape. A location where sufficient oxygen or another necessary target gas must be present is, for example, an enclosed container or other enclosed space.

The method according to the disclosure is carried out using such an arrangement and comprises the corresponding steps.

According to the disclosure, the first portable device and the first stationary device can detect the same target gas. Owing to the disclosure, the first portable device warns the user thereof if the target gas has appeared in at least one detection area at an impermissible concentration, i.e. at a concentration outside the first concentration range. In particular, the first portable device warns the user thereof if the target gas concentration in the detection area of the first stationary device is impermissible.

In addition, this feature makes it possible to check this and perform a plausibility check. It is possible for the first portable device to be brought into the vicinity of the first stationary device. It is also possible that the following situation may then occur: One device outputs an alarm but the other device does not, even though there is no fluid-tight wall or other shielding between the two devices. This situation is then an indication that a device is defective and is either incorrectly not outputting an alarm or is outputting a false alarm or a data connection between the two devices cannot be established or is interrupted.

According to the disclosure, the evaluation unit of the first portable device automatically decides whether the target gas concentration in the detection area of the first stationary device is outside the specified first concentration range. It is possible, but owing to this feature not necessary, that the first stationary device is also equipped as follows: The first stationary device also comprises an evaluation unit, wherein the evaluation unit of the first stationary device decides the following:

Is the target gas concentration in the detection area of the first stationary device within a specified concentration range? The first stationary device does not necessarily need to comprise such an evaluation unit. It is therefore not necessary to define a concentration range in a computer-evaluable manner and to transmit it to the first stationary device or to specify it in any other way to the first stationary device either. In many cases, this advantage makes it easier to use an arrangement according to the disclosure having a plurality of devices, wherein each device measures the respective concentration of the same target gas in a detection area while still ensuring that each measured target gas concentration is compared to a specified concentration range, in particular to the same concentration range. This in turn makes it easier to adapt the arrangement to changing specifications regarding a permissible target gas concentration and/or to different users.

In one embodiment, the arrangement comprises a further device in addition to the first stationary device. The further device can be a stationary device which can be positioned at an installation location or a portable device which a user can carry with him/her. If the further device is a stationary device, it does not necessarily comprise its own evaluation unit and its own output unit.

Just like the first stationary device, the further device also comprises a sensor unit and a communication unit and has a detection area. Just like the first portable device and the first stationary device, the further device is also able to measure the concentration of the target gas, specifically in the detection area of the further device. The first portable device, the first stationary device, and the further device thus measure the concentration of the same target gas in their respective detection area.

The arrangement can cause a message containing the target gas concentration measured by the further device to be transmitted to the first portable device. The evaluation unit of the first portable device can automatically decide whether the target gas concentration, which the further device has measured in its detection area, is outside the first concentration range. Overall, the evaluation unit can thus compare the three concentrations measured by the three devices in the three respective detection areas and relating to the same target gas to the same first concentration range.

The first portable device can generate the alarm by means of its output unit and output it in at least one form perceptible to a human if at least one of the following three events has occurred:
The target gas concentration, which the first portable device has measured in its detection area, is outside the first concentration range.
The target gas concentration, which the first stationary device has measured in its detection area, is outside the first concentration range.
The target gas concentration, which the further device has measured in its detection area, is outside the first concentration range.

This embodiment allows three different locations to be monitored for the presence of the same target gas using three different devices. The user of the first portable device is currently located at one location. The first stationary device is installed at one of the other locations (second location), and the further device is located at the third location. It is possible for the arrangement to comprise yet a further device, i.e. together a first portable device, a first stationary device, and two further devices, wherein the first portable device generates and outputs an alarm if the target gas concentration in at least one of these four detection areas is outside the first concentration range.

In one embodiment, the arrangement comprises a second portable device in addition to the first portable device. Just like the first portable device, the second portable device also comprises a sensor unit, a signal-processing evaluation unit, an output unit, and a communication unit and has a detection area. The second portable device can also be carried by a user, in particular attached to the user's personal protective equipment. The sensor unit of the second portable device can measure the concentration of the target gas in the detection area of the second portable device.

A data connection is or can be at least temporarily established between the first stationary device on the one hand and the first and second portable devices on the other. The arrangement can achieve the following: A message containing the target gas concentration measured by the first stationary device in its detection area is transmitted to both the first portable device and the second portable device via the corresponding data connection. Optionally, the message comprises information about the geoposition of the first stationary device.

The evaluation unit of the second portable device can automatically make the following two decisions:
Is the target gas concentration, which the second portable device has measured in its detection area, outside a specified second concentration range?
Is the target gas concentration, which the first stationary device has measured in its detection area, outside the second concentration range?

The second portable device can output an alarm in at least one form perceptible to a human by means of its output unit if at least one of the following two events has occurred:
The target gas concentration in the detection area of the second portable device is outside the second concentration range.
The target gas concentration in the detection area of the first stationary device is outside the second concentration range.

The second concentration range can coincide with the first concentration range or can differ from it.

This embodiment allows two portable devices to quasi (virtually, so as to speak) share the first stationary device. Both a user of the first portable device and a user of the second portable device will be alerted if the target gas concentration in the detection area of the first stationary device is impermissible. Again, both alarms relate to the same target gas. It is not necessary to use two different stationary devices for the two users, wherein these stationary devices measure the target gas concentration at practically the same installation location.

According to the disclosure, the first portable device generates an alarm and outputs the alarm if the target gas concentration measured by the first portable device and/or the target gas concentration measured by the first stationary device is outside the same specified first concentration range. According to the embodiment just described, the second portable device generates an alarm and outputs the alarm if the target gas concentration measured by the second portable device and/or the target gas concentration measured by the first stationary device is outside a defined second concentration range. These two concentration ranges can be the same or different. Two different concentration ranges are useful in the following situations, for example:
The target gas is harmful to humans. The user of the first portable device wears protective equipment which protects the user from this harmful target gas up to a certain target gas concentration, whereas the user of the second portable device does not wear such protective equipment.
One user's respiratory system is impaired, the other's is not. For example, one user suffers from asthma.
The target gas is oxygen or another target gas necessary for humans. The user of the first portable device carries a source of breathing air, such as a bottle of breathing air, and can therefore stay in an environment with a low oxygen concentration. The user of the second portable device does not carry such a source and therefore requires breathing air in his/her environment.

According to the disclosure, the first portable device generates an alarm and outputs the alarm if at least one of the following events has occurred:
The target gas concentration in the detection area of the first portable device is outside the first concentration range.
The target gas concentration in the detection area of the first stationary device is outside the first concentration range.

Owing to the disclosure, the first stationary device does not necessarily need to have its own evaluation unit and its own output unit.

In one embodiment, however, the first stationary device also comprises an output unit, wherein the output unit of the first stationary device can output an alarm in at least one form perceptible by a human, in particular acoustically. According to this embodiment, the event where the evaluation unit of the first portable device has detected an impermissible target gas concentration and therefore generated an alarm has the following effect:
The first portable device generates the alarm.
A message containing the generated alarm is transmitted from the first portable device to the first stationary device.
The output unit of the first stationary device outputs the alarm.
   The alarm output by the first portable device can have a form which is different from that of the first stationary device, e.g. haptic (portable device) vs. acoustic (stationary device).

Owing to this embodiment, people who are near the location where the first stationary device is installed will be alerted. It is possible, but owing to this embodiment not necessary, for the first stationary device to need to comprise its own evaluation unit or for the output unit of the first portable device to have to be able to alert other persons in addition to the user of the first portable device.

In one embodiment, the first portable device comprises an input unit. A user can use this input unit. The input unit is capable of recording a user input, wherein the user input specifies the first concentration range. In particular, the user can use the input unit to specify an upper and/or lower limit of the concentration range or to change a limit already specified and stored on the device. The first portable device uses this specification to generate and output the alarm. This embodiment allows the user of the first portable device to specify under which condition the first portable device generates an alarm. In particular, the user can specify the concentration range on the basis of the following potential criteria:
The target gas is harmful to humans. Is the user wearing protective equipment which protects the user to some extent from the harmful target gas?
The target gas is harmful to humans. Does the user spend all of his/her time outdoors or at least some of his time in an enclosed area? As a rule, a high target gas concentration lasts longer in an enclosed area than outdoors.
Is the user's respiratory system impaired?
The target gas is oxygen. Is the user carrying a source of oxygen or breathing air?

Owing to the embodiment just described, the same first portable device can be used for different applications. The embodiment therefore increases the flexibility with which the first portable device can be used.

An embodiment described below allows the user of the first portable device to decide whether he/she wants to be warned of an impermissible target gas concentration at a certain location or does currently not want to be warned. For example, an alarm is often not necessary and would only be distracting if the user is spending at least some of his/her time working in an enclosed space and the installation location of the first stationary device is outside this space. Therefore, the target gas currently cannot reach this space from the detection area of the first stationary device.

According to this embodiment, the first portable device can be operated either in a connected mode or in a local mode, optionally further in an idle (rest) state. Preferably, the user can use an actuating unit, such as a switch, to establish whether the first portable device is now to be operated in the connected mode or local mode.

In the connected mode, the first portable device will generate and output an alarm if the target gas is present in said device's own detection area or in the detection area of the first stationary device at a concentration which is outside the first concentration range. In the local mode, the first portable device will generate and output an alarm if the target gas is present in said device's own detection area at a concentration which is outside the first concentration range. In contrast, the first portable device does not output an alarm in the local mode regardless of the target gas concentration in the detection area of the first stationary device.

This embodiment can be combined with the embodiment described above, in which the arrangement comprises a further device, in particular a further stationary device. According to this combination, the first portable device will generate and output an alarm in the connected mode if the target gas is present in said device's own detection area or in the detection area of the first stationary device or in the detection area of the further device at a concentration which is outside the first concentration range. In the local mode, the first portable device will generate and output an alarm if the target gas is present in said device's own detection area or in the detection area of the first stationary device at a concentration which is outside the first concentration range. In contrast, the first portable device does not output an alarm in the local mode regardless of the target gas concentration in the detection area of the other device.

The following is an example application of the embodiment just described: The first stationary device is installed in the enclosed space just described, the other device is a stationary device and is positioned outside this space. The user sometimes works in the enclosed space and sometimes outside the space and near the installation location of the second stationary device. The user uses the first portable device in the local mode while working in the space and in the connected mode while working outside the space. In this application, the further stationary device can be installed in advance.

Preferably, the or each portable device and the or each stationary device comprise their own respective power supply unit. This eliminates the need to connect this device to a stationary power supply network, which is often not possible. In particular, the or a stationary device can, however, be at least temporarily connected to a stationary power supply network.

### BRIEF DESCRIPTION OF THE DRAWINGS

In the following, the disclosure is described on the basis of an exemplary embodiment. In the drawings,

FIG. 1 shows a first example embodiment with a user carrying a portable device, and three stationary devices;

FIG. 2 shows a second example embodiment with two users, each carrying a portable device, and a stationary device; and

FIG. 3 shows a third example embodiment with a user carrying a portable device, a stationary device in an enclosed space and a stationary device outside the enclosed space.

### DETAILED DESCRIPTION

In the exemplary embodiment, the arrangement according to the disclosure is used in a spatial area in which harmful gases can be present which gases are hazardous to humans. The harmful gases are, for example, flammable and/or toxic. As a rule, it is known which harmful gases may be present in the spatial area. The spatial area is monitored for the presence of these harmful gases. In the following, reference will be made to “the harmful gas” for short, which can also refer to different harmful gases. The harmful gas acts as the target gas of the exemplary embodiment.

The concentration range of the harmful gas is determined by an upper concentration limit. The harmful gas must not be present at a concentration above this upper concentration limit. The concentration range is therefore specified in such a way that a person is at risk, or at least can be at risk, if the target gas concentration is above this concentration limit, but is not at risk if the concentration is below the concentration limit. The concentration limit can vary from target gas to target gas.

A person working in this spatial area must be warned of the presence of the harmful gas. Therefore, on the one hand every person in the spatial area wears personal protective equipment and on the other a gas detection device on his/her personal protective equipment. This person will hereinafter be referred to as “the user” of the gas detection device. A gas detection device will hereinafter be referred to as “a device” for short.

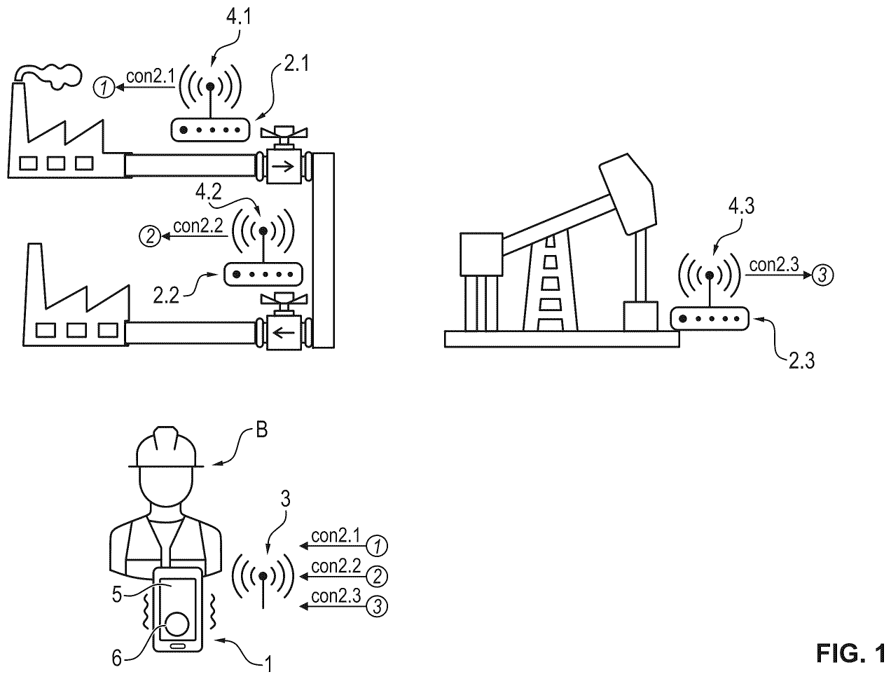

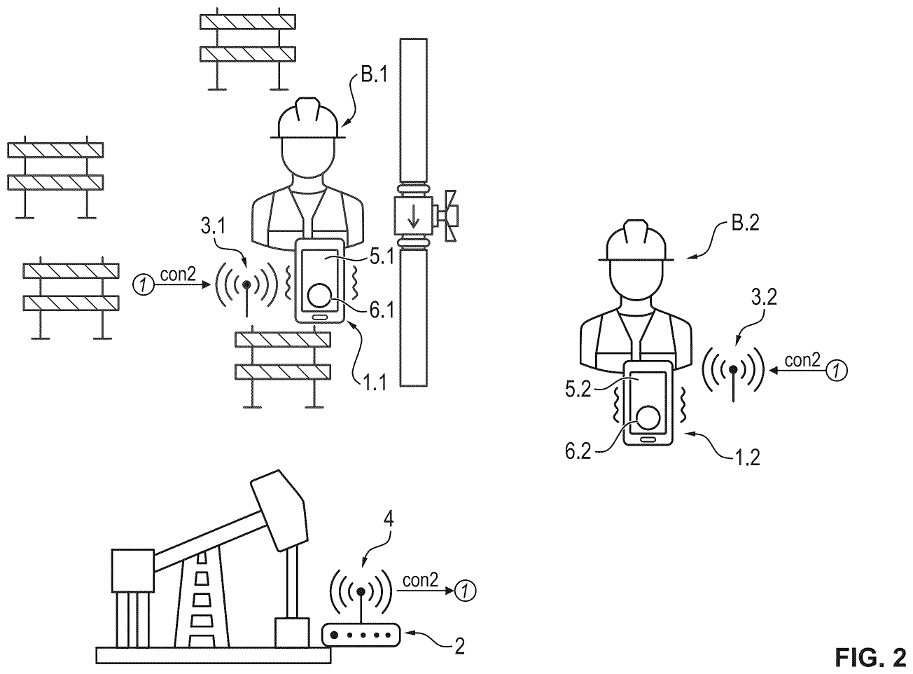

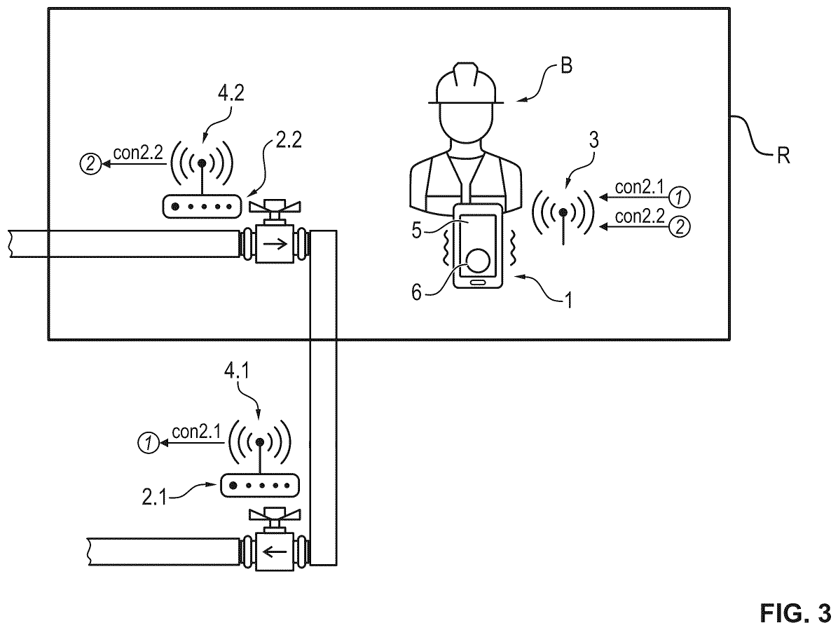

FIG. 1 and FIG. 3 show a user B wearing a device 1 on his/her protective equipment. FIG. 2 shows two users B.1, B.2 working in the same spatial area and each wearing a device 1.1, 1.2 on their respective protective equipment. A device 1, 1.1, 1.2 which a user B, B.1, B.2 wears on his/her protective equipment or can carry with him/her in another manner will hereinafter be referred to as a “portable device.” The portable device 1, 1.1, 1.2 belongs to the arrangement according to the exemplary embodiment of the disclosure.

In the exemplary embodiment, each portable device 1, 1.1, 1.2 comprises
a measuring chamber,
a sensor unit,
a signal-processing control unit having an evaluation unit,
an output unit 5, 5.1, 5.2,
a communication unit 3, 3.1, 3.2, and
its own power supply unit.

The measuring chamber is at least temporarily in fluid communication with the spatial area to be monitored and can receive a gas sample from the spatial area. The sensor unit can measure the concentration (the proportion, the content) of the or each harmful gas in a gas sample located in the measuring chamber, which gas is present or can be present in the spatial area, optionally the sum of the concentrations of all the harmful gases. For each possible harmful gas, the sensor unit generates, in one implementation, a signal which comprises information about the measured concentration of this harmful gas, and, in another implementation, a signal with the sum of the harmful gas concentrations.

In one embodiment, the sensor unit measures a detection variable which correlates with the desired harmful gas concentration, for example an electrical voltage, an electrical current, an electrical charge or an electrical resistance. The sensor unit applies a functional relationship, which is specified in a computer-evaluable form, to the value measured for the detection variable. The functional relationship describes the harmful gas concentration as a function of the detection variable. The application provides a value for the harmful gas concentration. Preferably, the sensor unit repeatedly performs the measurement, for example with a fixed sampling rate. The measurements provide at least one harmful gas signal.

The evaluation unit evaluates the or each harmful gas signal and is able to detect the or each harmful gas through this evaluation. “Detecting a harmful gas” means detecting the event in which the concentration of this harmful gas is outside a specified concentration range. Preferably, the sensor unit can measure the concentration of each harmful gas in the gas sample.

An identifier for the concentration range for the harmful gas is specified and stored in a computer-evaluable form, for example in a data memory of the portable device 1, 1.1, 1.2 or in a program wherein the evaluation unit executes this program. In particular, an upper concentration limit is stored.

In one embodiment, the portable device 1, 1.1, 1.2 comprises an input unit 6, 6.1, 6.2. Using this input unit 6, 6.1, 6.2, a user B, B. 1, B.2 can change a concentration range specified on the device, for example by selecting from a plurality of specified potential concentration ranges. For example, the user B, B.1, B.2 can increase the upper limit if the user B, B.1, B.2 is wearing appropriate protective equipment which protects him/her to a certain extent against a harmful gas and decrease it again if he/she is no longer wearing the protective equipment. The recorded specification of the user B, B.1, B.2 is preferably stored in a data memory of the device 1, 1.1, 1.2.

Different measuring principles for how a sensor unit measures the concentration of a harmful gas in a gas sample have become known. The sensor unit comprises, for example, a photoelectric or photoacoustic detector or a photoionization detector (PID) or a heat-tone sensor (catalytic sensor) or an electrochemical sensor. It is possible that the sensor unit comprises a plurality of sensors for different target gases, wherein these sensors are configured to operate according to different measuring principles. It is also possible for the user to carry a plurality of portable devices for different harmful gases. For short, reference will be made to “the user's portable device” below.

The control unit of the portable device 1, 1.1, 1.2 can generate an alarm if the evaluation unit has detected that the concentration of at least one harmful gas is above the lower concentration limit specified for this harmful gas.

The control unit can actuate the appropriate output unit 5, 5.1, 5.2 of a portable device 1, 1.1, 1.2. The actuated output unit 5, 5.1, 5.2 can output the alarm generated in at least one form perceptible by a human, in particular visually, acoustically, and/or haptically (through vibrations). Particularly preferably, the output unit 5, 5.1, 5.2 causes the device 1, 1.1, 1.2 to vibrate. A user B, B.1, B.2 often perceives vibrations even when there is a lot of noise in the area around him/her and he/she is not looking at the output unit 5, 5.1, 5.2 and/or it is relatively dark or foggy or smoky. Optionally, the output unit 5, 5.1, 5.2 outputs the concentration measured for each possible harmful gas, even if the concentration of a harmful gas is below the concentration limit.

The power supply unit of the portable device 1, 1.1, 1.2 supplies its electrical consumers with electrical energy. As a result of having its own power supply unit, it is not necessary to connect the portable device 1, 1.1, 1.2 to a stationary power supply network, at least not permanently. This would normally not even be possible during use.

A portable device 1, 1.1, 1.2 which the user B, B.1, B.2 wears on his/

her personal protective equipment is able to examine a gas sample from the area directly around the user B, B.1, B.2 for the presence of potential harmful gases and, in doing so, to preferably measure the concentration of each potential harmful gas. The area around the user B, B.1, B.2 from which the analyzed gas sample originates is referred to as “the detection area” of the portable device 1, 1.1, 1.2. The portable device 1, 1.1, 1.2 can therefore warn the user B, B.1, B.2 of the presence of a harmful gas within a sufficiently short response time if this harmful gas is present in the relevant detection area around the user B, B.1, B.2. As long as the portable device 1, 1.1, 1.2 has not detected any harmful gas with an impermissible concentration in its detection area, there is currently no danger for the user B, B.1, B.2.

However, it is possible for a harmful gas to escape from a location outside the detection area of the portable device 1, 1.1, 1.2. The portable device 1, 1.1, 1.2 is therefore unable to warn the user B, B.1, B.2 about this harmful gas not at all or not quickly enough. However, the escaping harmful gas can be conveyed around the user B, B.1, B.2 by a natural or artificially generated air flow or by diffusion into the detection area. It is also possible that the user B, B.1, B.2 quickly proceeds to an area in which a harmful gas is present, in particular into a container or enclosed space. The portable device 1, 1.1, 1.2 is able to detect this harmful gas, but only when this harmful gas has entered the detection area of the portable device 1, 1.1, 1.2, i.e. around the user B, B.1, B.2. In addition, sometimes a time period, which is also referred to as the response time of the device, inevitably elapses between the event that a harmful gas is present in the vicinity of the portable device 1, 1.1, 1.2 and the event that the portable device 1, 1.1, 1.2 detects this harmful gas and generates and outputs an alarm. This time period is determined in particular by the time required for the gas sample to reach the measuring chamber and/or by the response time of the sensor unit.

It is desirable for the user B, B.1, B.2 to be given earlier a warning, i.e. as soon as the escaped harmful gas is still outside the detection area of the user's own portable device 1, 1.1, 1.2. For this purpose, the arrangement therefore comprises at least one device, preferably a plurality of devices, which can each be installed at a location in the spatial area to be monitored.

To distinguish it from the portable device 1, 1.1, 1.2 just described, such a device will be referred to as the “stationary device.” The term “stationary” means that the device is stationary during use and does not move when the user B, B.1, B.2 moves. As a rule, a stationary device can also be worn by the user B, B.1, B.2 or by another person, but not necessarily attached to his/her personal protective equipment. Preferably, a user B, B.1, B.2 can install each stationary device at a location, carry out work in the spatial area to be monitored and, after completion of the work, remove the device and, for example, install it somewhere else. The location where a stationary device is currently located is referred to as the “installation location.”

FIG. 1 shows three stationary devices 2.1, 2.2, 2.3 as examples; FIG. 2 shows one stationary device 2, and FIG. 3 shows two stationary devices 2.1, 2.2. In the example of FIG. 3, the stationary device 2.2 is located at an installation location in an enclosed space R, and the stationary device 2.1 is located at an installation location outside the enclosed space R.

Each stationary device 2, 2.1, 2.2, 2.3 comprises
a measuring chamber which is at least temporarily in fluid communication with the spatial area to be monitored,
a sensor unit,
a communication unit 4, 4.1, 4.2, 4.3, and
preferably its own power supply unit.

A stationary device 2, 2.1, 2.2, 2.3 does not necessarily comprise its own evaluation unit for deciding whether a measured harmful gas concentration lies outside a specified concentration range. Furthermore, a stationary device 2, 2.1, 2.2, 2.3 does not necessarily comprise an own output unit for outputting a measured harmful gas concentration and/or an alarm. However, as a rule, a stationary device 2, 2.1, 2.2, 2.3 comprises at least one indicator light, optionally a plurality of indicator lights, for indicating proper operation and/or a fault.

It is possible that a stationary device 2, 2.1, 2.2, 2.3 can be connected to a stationary power supply network. In one implementation, a stationary device 2, 2.1, 2.2, 2.3 can be connected to the stationary power supply network and additionally comprises its own power supply unit. If the stationary device 2, 2.1, 2.2, 2.3 is connected to a stationary power supply network, the electrical consumers of the stationary device 2, 2.1, 2.2, 2.3 are supplied with power from the stationary power supply network, otherwise from its own power supply unit.

The measuring chamber of the or each stationary device 2, 2.1, 2.2, 2.3 is also able to receive a gas sample. The sensor unit of the or each stationary device 2, 2.1, 2.2, 2.3 can measure the concentration con2, con2.1, con2.2, con2.3 of at least one harmful gas, which is or can be present in the spatial area, in a gas sample in the measuring chamber. In one embodiment, the sensor unit can measure the respective concentration of each potential harmful gas in the spatial area. In another embodiment, the arrangement comprises at least two different stationary devices 2, 2.1, 2.2, 2.3 for at least two different harmful gases. It is possible that the stationary devices 2, 2.1, 2.2, 2.3 of the arrangement are collectively able to detect every possible harmful gas. It is also possible that the stationary devices 2, 2.1, 2.2, 2.3 are collectively only able to detect some of the possible harmful gases, specifically the most dangerous harmful gases.

Just like the portable device 1, 1.1, 1.2, the or each stationary device 2, 2.1, 2.2, 2.3 has a detection area around the stationary device or around the free end of a fluid-guiding unit guiding into its measuring chamber. The installation location of a stationary device 2, 2.1, 2.2, 2.3 is selected in one application such that there is at least one component, for example a valve, a pipeline, a branch or where a plurality of pipelines are connected, in the detection area of the stationary device 2, 2.1, 2.2, 2.3, from which component a harmful gas can emerge. In another application, the installation location is in or on an escape route, for example near an escape door or an escape chamber. These two applications can be combined.

The sensor unit can analyze a gas sample from this detection area around the stationary device 2, 2.1, 2.2, 2.3. For each harmful gas whose concentration the sensor unit can measure, the sensor unit generates a harmful gas signal which comprises a concentration above the measured harmful gas concentration con2, con2.1, con2.2, con2.3.

It is possible that at least one stationary device 2, 2.1, 2.2, 2.3 additionally comprises an evaluation unit. The optional evaluation unit of the stationary device 2, 2.1, 2.2, 2.3 can also detect the event that the concentration con2, con2.1, con2.2, con2.3 measured for a harmful gas is above the upper concentration limit for this harmful gas. For this purpose, the evaluation unit evaluates the relevant harmful gas signal.

A data connection via radio waves is permanently or at least temporarily established between a stationary device 2, 2.1, 2.2, 2.3 and a portable device 1, 1.1, 1.2, preferably by means of a conventional and/or standardized transmission protocol. For this data connection, the communication unit 4, 4.1, 4.2, 4.3 of the stationary device 2, 2.1, 2.2, 2.3 and the communication unit 3, 3.1, 3.2 of the portable device 1, 1.1, 1.2 are used.

In one implementation, this data connection is permanently established-of course only if the distance between the portable device 1, 1.1, 1.2 and the stationary device 2, 2.1, 2.2, 2.3 is not too large and the transmission of radio waves is not prevented by shielding. In another embodiment, a portable device 1, 1.1, 1.2 queries the or each stationary device 2, 2.1, 2.2, 2.3, preferably with a fixed query frequency, and the data connection is temporarily established and then interrupted again.

A message is transmitted from the stationary device 2, 2.1, 2.2, 2.3 to the portable device 1, 1.1, 1.2 via the data connection. This message comprises the harmful gas signal for the or each harmful gas which the sensor unit of the stationary device 2, 2.1, 2.2, 2.3 has measured. This harmful gas signal comprises information about each of the measured harmful gas concentrations con2, con2.1, con2.2, con2.3. The evaluation unit of the portable device 1, 1.1, 1.2 receives the harmful gas signal and checks whether the measured and transmitted concentration con2, con2.1, con2.2, con2.3 of this harmful gas is above the upper concentration limit. This implementation does not necessarily require the stationary device 2, 2.1, 2.2, 2.3 to have its own evaluation unit. Optionally, the message also comprises a time stamp and/or an identification of the corresponding geoposition of the stationary device 2, 2.1, 2.2, 2.3.

In this implementation, the evaluation unit of the portable device 1, 1.1, 1.2 thus processes at least two different harmful gas signals for at least one possible harmful gas, specifically
a harmful gas signal from the sensor unit of the portable device 1, 1.1, 1.2 itself, and
a harmful gas signal transmitted from the sensor unit of the or at least one stationary device 2, 2.1, 2.2, 2.3.

The evaluation unit of the portable device 1, 1.1, 1.2 generates an alarm if at least one harmful gas signal indicates a concentration above the upper concentration limit for this harmful gas. The output unit 5, 5.1, 5.2 of the portable device 1, 1.1, 1.2 outputs an alarm in response to this detection. Preferably, the evaluation unit 5, 5.1, 5.2 additionally outputs the information as to whether this impermissible (excessive) harmful gas concentration con2, con2.1, con2.2, con2.3 is present in the detection area of the portable device 1, 1.1, 1.2 or in the detection area of the stationary device 2, 2.1, 2.2.

In a different implementation, the stationary device 2, 2.1, 2.2, 2.3 also comprises its own evaluation unit which is able to compare a measured target gas concentration with an upper concentration limit. The stationary device 2, 2.1, 2.2, 2.3 itself is therefore able to detect a harmful gas. If the stationary device 2, 2.1, 2.2, 2.3 has detected a harmful gas, the evaluation unit generates an alarm.

The stationary device 2, 2.1, 2.2, 2.3 transmits a message with this alarm to the or at least one portable device 1, 1.1, 1.2, and the output unit of the portable device 1, 1.1, 1.2 outputs this alarm as described above. The different implementation does not necessarily require that the harmful gas signal containing the harmful gas concentration con2, con2.1, con2.2, con2.3 is transmitted to the portable device 1, 1.1, 1.2. Furthermore, the different implementation does not require that a permanent data connection be established between the stationary device 2, 2.1, 2.2, 2.3 and the portable device 1, 1.1, 1.2. Rather, it is sufficient if this data connection is established or will be established when the stationary device 2, 2.1, 2.2, 2.3 has detected an impermissible (excessive) harmful gas concentration.

In a further implementation, the or at least one stationary device 2, 2.1, 2.2, 2.3 comprises an output unit which can output an alarm, but no evaluation unit. The stationary device 2, 2.1, 2.2, 2.3 transmits the harmful gas concentration con2, con2.1, con2.2, con2.3 which it has measured to the or at least one portable device 1, 1.1, 1.2. The evaluation unit of this portable device 1, 1.1, 1.2 decides whether the harmful gas concentration is permissible or impermissible. If the harmful gas concentration is impermissible, the portable device 1, 1.1, 1.2 generates an alarm. This alarm is again transmitted to the stationary device 2, 2.1, 2.2, 2.3, and the output unit of the stationary device 2, 2.1, 2.2, 2.3 outputs the received alarm. This embodiment reduces the risk of a user B, B.1, B.2 not perceiving an alarm, but does not require that a concentration limit be stored and kept up-to-date in the stationary device 2, 2.1, 2.2, 2.3.

At least two of these implementations can be combined with one another. On the one hand, at least one harmful gas signal is thus continuously transmitted to the portable device 1, 1.1, 1.2 and, on the other hand, an alarm is transmitted thereto, specifically when the stationary device 2, 2.1, 2.2, 2.3 has detected a harmful gas. This embodiment creates intentional redundancy.

In an example embodiment, the portable device 1, 1.1, 1.2 sends at regular intervals a request message. Each stationary device, 2, 2.1, 2.2, 2.3 which has received this request message sends a response message. This response message preferably comprises a unique identifier and/or information about the geoposition (installation location) of the stationary device 2, 2.1, 2.2, 2.3 as well as a time stamp. The portable device 1, 1.1, 1.2 outputs the information about which stationary devices have responded. The portable device 1, 1.1, 1.2 generates and outputs an error message if a stationary device 2, 2.1, 2.2, 2.3 has not responded to a request message within a given time period.

Optionally, the evaluation unit of the portable device 1, 1.1, 1.2 determines how far away a stationary device 2, 2.1, 2.2, 2.3 which has sent a response message is from the portable device 1, 1.1, 1.2. It is possible that the evaluation unit additionally determines the direction to the stationary device 2, 2.1, 2.2, 2.3. The output unit 5, 5.1, 5.2 then additionally outputs the distance determined and optionally the direction in a form perceptible to a human.

So far, an embodiment has been described in which a data connection is permanently or at least temporarily established between a stationary device 2, 2.1, 2.2, 2.3 and a portable device 1, 1.1, 1.2 and this data connection can be used to transmit a harmful gas signal and/or an alarm to the portable device 1, 1.1, 1.2. A variant will be described below.

FIG. 1 and FIG. 3 show a first embodiment in which two stationary devices 2.1, 2.2 are installed at two different installation locations. In this embodiment, the two installation locations are chosen so that there is a valve in each pipeline in the two detection areas. A respective data connection is permanently or at least temporarily established between the two stationary devices 2.1, 2.2 and the portable device 1. Each stationary device 2.1, 2.2 can transmit a harmful gas signal and/or an alarm to the same portable device 1 via this data connection. The two harmful gas signals and the alarm can relate to the same harmful gas or to two different harmful gases. In the example of FIG. 3, the installation location of the stationary device 2.2 is located in an enclosed space R; the installation location of the stationary device 2.1 is located outside this space R. Currently, the user B with the portable device 1 is located in the space R.

FIG. 2 shows an embodiment in which two users B.1, B.2 are working at two different locations and are each carrying a portable device 1.1, 1.2. A data connection is established between the same stationary device 2 and each of the two portable devices 1.1, 1.2, either permanently or at least temporarily. The stationary device 2 can transmit the same harmful gas signal and optionally also the same alarm and/or its own geoposition and/or identifier to both portable devices 1.1, 1.2. The two users B.1, B.2 are thus informed simultaneously that a harmful gas has been detected in the detection area of the stationary device 2.

In the embodiments just described, the portable device 1, 1.1, 1.2 which a user B, B.1, B.2 is wearing on his personal protective equipment always informs the user B, B.1, B.2 about a result which the or a stationary device 2, 2.1, 2.2 has achieved, provided that a data connection is established between the stationary device 2, 2.1, 2.2 and the portable device 1, 1.1, 1.2. At a minimum, however, the portable device informs the user B, B.1, B.2 of any alarm created and transmitted by the or a stationary device 2, 2.1, 2.2.

In an alternative embodiment, a user B, B.1, B.2 can temporarily prevent a stationary device 2, 2.1, 2.2, 2.3 from informing him/her about an alarm or about a harmful gas concentration con2, con2.1, con2.2, con2.3. For example, the user B, B.1, B.2 is working in an enclosed space R, and the detection area of his/her portable device 1, 1.1, 1.2 covers a sufficiently large part of the enclosed space R. In this alternative embodiment, the portable device 1, 1.1, 1.2 preferably comprises a switch. Using this switch, the user B, B.1, B.2 can specify whether he/she wants to be informed by a stationary device 2, 2.1, 2.2, 2.3 about a harmful gas concentration and/or about an alarm or whether this information should not be provided. The user B, B.1, B.2 can thus establish whether the portable device 1, 1.1, 1.2 is to be operated in a connected or in a local mode.

If the user B, B.1, B.2 has selected the connected mode, his/her portable device 1, 1.1, 1.2 informs him/her not only about an excessive harmful gas concentration in the detection area of the portable device 1, 1.1, 1.2, but also about an excessive harmful gas concentration con2, con2.1, con2.2, con2.3 in the detection area of the or at least one stationary device 2, 2.1, 2.2, 2.3. In the local mode, the user's portable device 1, 1.1, 1.2 informs him/her about an excessive harmful gas concentration in the detection area of the portable device 1, 1.1, 1.2, but not about an excessive harmful gas concentration con2, con2.1, con2.2, con2.3 in the detection area of the or at least one stationary device 2, 2.1, 2.2, 2.3.

An implementation of this alternative embodiment is described below. The user B, B.1, B.2 assigns at least one stationary device to his/her portable device 1, 1.1, 1.2. For example, the user B in FIG. 1 assigns the two stationary devices 2.1, 2.2 to his/her portable device 1. An identifier for each assigned stationary device 2.1, 2.2 is stored in a data memory of the portable device 1. The user B can choose between the following two alternatives:
Each stationary device 2, 2.1, 2.2, 2.3 can transmit a harmful gas signal and/or an alarm to the portable device 1, including a stationary device 2 which is not assigned to the portable device 1 of the user B.
Only a stationary device 2.1, 2.2 assigned to the portable device 1 can transmit a harmful gas signal and/or an alarm to the portable device 1, but not another stationary device 2.3.

FIG. 3 shows a third example embodiment. If the user B has selected the connected mode, his/her portable device 1 also outputs an alarm if an excessive harmful gas concentration con2.1, con2.2 is present in the detection area of a stationary device 2.1, 2.2. The two harmful gas concentrations measured by the two stationary devices 2.1, 2.2 are transmitted to the portable device 1. In the local mode, the portable device 1 of the user B outputs an alarm if an excessive harmful gas concentration con2.2 is present in the detection area of the portable device 1 or in the detection area of the stationary device 2.2 in the space R, but not if an excessive harmful gas concentration is present in the detection area of the stationary device 2.1 outside the space R.

In one implementation, the portable device 1 automatically detects that the stationary device 2.2 is located in the same space R as the portable device 1, while the stationary device 2.1 is located outside this space R. For example, the portable device 1 evaluates the strength of a signal from the two stationary devices 2.1, 2.2 in order to make this decision. Optionally, the portable device 1 additionally evaluates the transmitted geoposition of each stationary device 2.1, 2.2.

### LIST OF REFERENCE SIGNS

| | |
|---|---|
| 1, 1.1, 1.2 | portable device, is carried by the user B, B.1, B.2, |
| | comprises the communication unit 3, 3.1, 3.2 and the |
| | output unit 5, 5.1, 5.2 |
| 2, 2.1, 2.2, | stationary device, measures the harmful gas concentration |
| 2.3 | con2, con2.1, con2.2, con2.3, comprises the |
| | communication unit 4, 4.1, 4.2, 4.3 |
| 3, 3.1, 3.2 | communication unit of the portable device 1, 1.1, 1.2 |
| 4, 4.1, 4.2, 4.3 | communication unit of the stationary device 2, 2.1, |
| | 2.2, 2.3 |
| 5, 5.1, 5.2 | output unit of the portable device 1, 1.1, 1.2, outputs |
| | an alarm visually, acoustically and/or haptically |
| 6, 6.1, 6.2 | input unit of the portable device 1, 1.1, 1.2, |
| | allows a user B, B.1, B.2 to specify a concentration |
| | range for the evaluation unit of the portable device |
| | 1, 1.1, 1.2 |
| B, B.1, B.2 | user, wears the portable device 1, 1.1, 1.2 on his/her |
| | personal protective equipment |
| con2, con2.1, | harmful gas concentration measured by the stationary |
| con2.2, con2.3 | device 2, 2.1, 2.2, 2.3 |
| R | enclosed space in which the stationary device 2.2 is |
| | installed and in which the user B is located |

1-10. (canceled)

11. An arrangement comprising:
a first portable gas detection device; and
a first stationary gas detection device,
wherein the first portable gas detection device:
comprises a sensor unit, an output unit, a signal-processing evaluation unit, and a communication unit;
has a detection area; and
is configured to be carried by a user;
wherein the first stationary gas detection device:
comprises a sensor unit and a communication unit;
has a detection area; and
is configured to be positioned at an installation location;
wherein the sensor unit of the first portable gas detection device is configured to measure a first concentration of a target gas in the detection area of the first portable gas detection device,
wherein the sensor unit of the first stationary gas detection device is configured to measure a second concentration of the target gas in the detection area of the first stationary gas detection device,
wherein the communication unit of the first portable gas detection device and the communication unit of the first stationary gas device are configured to at least temporarily establish a data connection between the first portable gas detection device and the first stationary gas detection device,
wherein the arrangement is configured to transmit, via the data connection, the measured second concentration of the target gas to the first portable gas detection device,
wherein the evaluation unit of the first portable gas detection device is configured to automatically determine:
whether the first concentration is outside a given first concentration range; and
whether the measured second concentration is outside the first concentration range, and
wherein the first portable gas detection device is configured to:
generate an alarm, wherein the alarm is generated in response to at least one of:
the first concentration being outside the first concentration range; or
the measured second concentration being outside the first concentration range; and
output the alarm by means of the output unit of the first portable gas detection device in at least one form perceptible by a human.

12. The arrangement of claim 11, wherein:
the arrangement comprises an additional gas detection device,
wherein the additional gas detection device:
comprises a sensor unit and a communication unit; and
has a detection area,
wherein the sensor unit of the additional gas detection device is configured to measure a third concentration of the target gas in the detection area of the additional gas detection device,
wherein the communication unit of the first portable gas detection device and the communication unit of the additional gas detection device are configured to at least temporarily establish a data connection between the first portable gas detection device and the additional gas detection device,
wherein the arrangement is configured to transmit, via the data connection between the first portable gas detection device and the additional gas detection device, the measured third concentration of the target gas measured by the additional gas detection device to the first portable gas detection device,
wherein the evaluation unit of the first portable gas detection device is additionally configured to automatically determine whether the measured third concentration is outside the first concentration range, and
wherein the first portable gas detection device is configured to generate and output the alarm if at least one of the following concentrations is outside the first concentration range:
the first concentration, or
the measured second concentration, or
the measured third concentration.

13. The arrangement of claim 12, wherein:
the additional gas detection device:
is a stationary gas detection device and is configured to be positioned at an installation location, or
is a mobile gas detection device and is configured to be carried by a user.

14. The arrangement of claim 11, wherein:
the arrangement further comprises a second portable gas detection device,
wherein the second portable gas detection device:
comprises a sensor unit, an output unit, a signal-processing evaluation unit, and a communication unit;
has a detection area; and
is configured to be carried by a user,
wherein the sensor unit of the second portable gas detection device is configured to measure a third concentration of the target gas in the detection area of the second portable gas detection device,
wherein the communication unit of the second portable gas detection device and the communication unit of the first stationary gas detection device are configured to at least temporarily establish a data connection between the second portable gas detection device and the first stationary gas detection device,
wherein the arrangement is configured to transmit, via the data connection between the second portable gas detection device and the first stationary gas detection device, the measured second concentration of the target gas to the second portable gas detection device,
wherein the evaluation unit of the second portable gas detection device is configured to automatically determine:
whether the measured third concentration is outside a given second concentration range; and
whether the measured second concentration is outside the second concentration range,
wherein the second portable gas detection device is configured to generate a second alarm and to output, by using the output unit of the second portable gas detection device, the second alarm in at least one form perceptible to a human in response to at least one of:
the measured third concentration being outside the second concentration range; or
the measured second concentration being outside the second concentration range.

15. The arrangement of claim 11, wherein:
the first portable gas detection device further comprises an input unit,
wherein the input unit is configured to capture a user input specifying the first concentration range, and
wherein the first portable gas detection device is configured to generate and output the alarm in response to at least one of:
the first concentration being outside the captured first concentration range; or
the measured second concentration being outside the captured first concentration range.

16. The arrangement according to claim 11, wherein:
the first portable gas detection device is configured to be operated either:
in a connected mode, or
in a local mode,
wherein the first portable gas detection device is configured to generate and output the alarm, while operating in the connected mode, by means of the output unit of the first portable gas detection device, in response to at least one of:
the first concentration being outside the first concentration range, or
the measured second concentration being outside the first concentration range, and
wherein the first portable gas detection device is configured to generate and output the alarm, while operating in the local mode, by means of the output unit of the first portable gas detection device, if the first concentration is outside the first concentration range.

17. The arrangement of claim 16, wherein:
the arrangement comprises a second stationary gas detection device with a sensor unit,
wherein the second stationary gas detection device is positioned at an installation location in an enclosed space,
wherein the sensor unit of the second stationary gas detection device is configured to measure a third concentration of the target gas in the detection area of the second stationary gas device,
wherein the first stationary gas detection device is positioned at an installation location outside the enclosed space,
wherein the first portable gas detection device is configured to generate and output the alarm, while operating in the connected mode by means of its output unit, in response to at least one of:
the first concentration being outside the first concentration range;
the measured second concentration being outside the first concentration range; or
the measured third concentration being outside the first concentration range, and
wherein the first portable gas detection device is configured to generate and output the alarm, when operating in the local mode, by means of its output unit, in response to at least one of:
the first concentration being outside the first concentration range; or
the measured third target being outside the first concentration range.

18. The arrangement of claim 11, wherein the alarm comprises a haptic alarm.

19. The arrangement of claim 11, wherein the alarm comprises an acoustic alarm.

20. The arrangement of claim 11, wherein:
the first portable gas detection device is configured to send, to the first stationary gas device and with a predetermined repetition frequency, a request message,
wherein the first portable gas detection device is further configured to output an error message if a given threshold time period expires without receiving a response to the request message.

21. A method for monitoring a spatial area for the presence of a target gas:
wherein the method is carried out using an arrangement,
wherein the arrangement comprises:
a first portable gas detection device; and
a first stationary gas detection device,
wherein the first portable gas detection device:
comprises a sensor unit, an output unit, a signal-processing evaluation unit, and a communication unit; and
has a detection area,
wherein the first stationary gas detection device:
comprises a sensor unit and a communication unit; and
has a detection area,
wherein the method is carried out while:
the first portable gas detection device is carried by a user; and
the first stationary gas detection device is positioned at an installation location, and
wherein the method comprises the steps of:
with the sensor unit of the first portable gas detection device, measuring a first concentration of the target gas in the detection area of the first portable gas detection device;
with the sensor unit of the first stationary gas detection device, measuring a second concentration of the target gas in the detection area of the first stationary gas detection device;
with the communication unit of the first portable gas detection device and the communication unit of the first stationary gas device, at least temporarily establishing a data connection between the first portable gas detection device and the first stationary gas detection device;
transmitting, via the data connection, the measured second concentration of the target gas to the first portable gas detection device;
with the evaluation unit of the first portable gas detection device, automatically determining:
whether the first concentration is outside a given first concentration range; and
whether the measured second concentration is outside the first concentration range;
generating, by the first portable gas detection device, an alarm in response to at least one of:
the first concentration being outside the first concentration range, or
the measured second concentration being outside the first concentration range; and
outputting, by the output unit of the first portable gas detection device, the alarm in at least one form perceptible by a human.

22. The method of claim 21, wherein:
the arrangement comprises an additional gas detection device,
wherein the additional gas detection device:
comprises a sensor unit and a communication unit; and
has a detection area,
wherein the method comprises the further steps of:
with the sensor unit of the additional gas detection device, measuring a third concentration of the target gas in the detection area of the additional gas detection device;
establishing, using the communication unit of the first portable gas detection device and the communication unit of the additional gas detection device, a data connection between the first portable gas detection device and the additional gas detection device;
transmitting, via the data connection between the first portable gas detection device and the additional gas detection device, the measured third concentration of the target gas to the first portable gas detection device; and
with the evaluation unit of the first portable gas detection device, automatically determining whether the measured third concentration is outside the first concentration range,
wherein the first portable gas detection device generates and outputs the alarm if at least one of the following concentrations is outside the first concentration range;
the first concentration, or
the measured second concentration, or
the measured third concentration.

23. The method of claim 22, wherein:
the additional gas detection device:
is a stationary gas detection device and is configured to be positioned at an installation location, or
is a mobile gas detection device and is configured to be carried by a user.

24. The method of claim 21, wherein:
the arrangement further comprises a second portable gas detection device,
wherein the second portable gas detection device:
comprises a sensor unit, an output unit, a signal-processing evaluation unit, and a communication unit;
has a detection area; and
is configured to be carried by a user,
wherein the method further comprises the steps of:
with the sensor unit of the second portable gas detection device, measuring a third concentration of the target gas in the detection area of the second portable gas detection device;
with the communication unit of the second portable gas detection device and the communication unit of the first stationary gas detection device, at least temporarily establishing a data connection between the second portable gas detection device and the first stationary gas detection device;
transmitting, via the data connection between the second portable gas detection device and the first stationary gas detection device, the measured second concentration of the target gas to the second portable gas detection device,
with the evaluation unit of the second portable gas detection device, automatically determining:
whether the measured third concentration is outside a given second concentration range; and
whether the measured second concentration is outside the second concentration range,
generating, by the second portable gas detection device, a second alarm and to output, wherein the second alarm is generated by using the output unit of the second portable gas detection device and wherein the second alarm is outputted in at least one form perceptible to a human in response to at least one of:
the measured third concentration being outside the second concentration range; or
the measured second concentration being outside the second concentration range.

25. The method of claim 21, wherein:
the first portable gas detection device further comprises an input unit,
wherein the method further comprises capturing, with the input unit, a user input specifying the first concentration range, and
wherein the first portable gas detection device is configured to generate and output the alarm in response to at least one of:
the first concentration being outside the captured first concentration range; or
the measured second concentration being outside the captured first concentration range.

26. The method of claim 21, wherein
the first portable gas detection device is operated, at different times:
in a connected mode; and
in a local mode,
wherein the first portable gas detection device, while operating in the connected mode, generates the alarm and outputs the alarm by means of the output unit of the first portable gas detection device in response to at least one of:
the first concentration being outside the first concentration range; or
the measured second concentration being outside the first concentration range, and
wherein the first portable gas detection device, while operating in the local mode, generates the alarm and outputs the alarm, by means of the output unit of the first portable gas detection device, if the first concentration is outside the first concentration range.

27. The method of claim 26, wherein:
the arrangement comprises a second stationary gas detection device with a sensor unit,
wherein the second stationary gas detection device is positioned at an installation location in an enclosed space,
wherein the first stationary gas detection device is positioned at an installation location outside the enclosed space,
wherein the sensor unit of the second stationary gas detection device measures a third concentration of the target gas in the detection area of the second stationary gas device,
wherein the first portable gas detection device, while operating in the connected mode, generates the alarm and outputs the alarm by means of its output unit in response to at least one of:
the first concentration being outside the first concentration range;
the measured second concentration being outside the first concentration range; or
the measured third concentration being outside the first concentration range, and
wherein the first portable gas detection device, when operating in the local model, generates the alarm and outputs the alarm by means of its output unit in response to at least one of:
the first concentration being outside the first concentration range; or
the measured third concentration being outside the first concentration range.

28. The method of claim 21, wherein the alarm comprises a haptic alarm.

29. The method of claim 21, wherein the alarm comprises an acoustic alarm.

30. The method of claim 21, further comprising:
sending, by the first portable gas detection device and to the first stationary gas device with a predetermined repetition frequency, a request message; and
if a given threshold time period expires without receiving a response to the request message is received, outputting an error message.
